(19) United States
(12) Patent Application Publication
(10) Pub. No.: US 2009/0036516 A1
(43) Pub. Date: Feb. 5, 2009

(54) COMPOUNDS FOR TREATING METABOLIC SYNDROME

(75) Inventors: Urs Scherrer, Riex (CH); Anna Sparatore, Milan (IT)

Correspondence Address: SCHNECK & SCHNECK, P.O. BOX 2-E, SAN JOSE, CA, 95109-0005, US

(73) Assignee: CTG PHARMA S.R.L., Milan (IT)

(21) Appl. No.: 11/722,406

(22) PCT Filed: Dec. 21, 2005
(86) PCT No.: PCT/EP05/13777

§ 371 Date: Sep. 4, 2008

Related U.S. Application Data
(60) Provisional application No. 60/439,714, filed on Jan. 13, 2003.

(51) Int. Cl. A61K 31/385; C07C 321/00; C07D 339/06; A61P 29/00; C07D 339/04; A61K 31/235

(52) U.S. Cl. 514/441; 560/17; 514/533; 514/440; 549/39; 549/37

(57) ABSTRACT

Compounds of general formula:

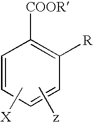

wherein R′, R, x and z have the meaning reported in the specification, are useful for treating inflammatory diseases including metabolic syndrome, diabetes, obesity, dyslipidemia, and insulin resistance.

### BACKGROUND OF THE INVENTION

Metabolic Syndrome, a newly recognized clinical entity, is also called the “deadly quartet” of insulin resistance, dyslipidemia (lipid abnormalities), arterial hypertension, and obesity. It is an ubiquitous disease of Western civilization, both highly prevalent and readily treatable. In USA, almost 25% of the adult population is estimated to display the metabolic syndrome, and its incidence increases to roughly 60% in obese individuals. This syndrome is more common in men than in women and its prevalence increases with age. Metabolic syndrome significantly increases the risk of developing cardiovascular disease (3-4 times), and greatly increases the risk of developing diabetes (up to 25 times) and aggravates its associated complications.

The metabolic syndrome also is associated with an increased incidence of other diseases, like cancer (particularly colon cancer) and Alzheimer's disease. In view of the relevant pathogenic role played by inflammation (see below), several diseases characterized by inflammation are also associated with this syndrome.

Metabolic syndrome is characterized by a tissue resistance to insulin that is reflected by augmented insulin plasma concentration and/or impaired insulin stimulation of glucose uptake in the body.

To date, no treatment appears to be satisfactory, because of a poor efficacy and/or limited tolerability.

### FIELD OF THE INVENTION

The present invention relates to the field of pharmacology, and to novel compounds for the pharmacological treatment of metabolic syndrome, said compounds having the following general formula (I):

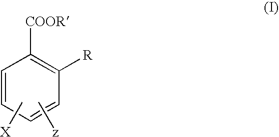

wherein:

### R=—OCOCH₃, —OH

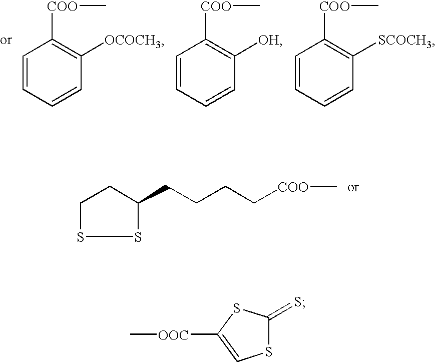

R′ is H, methyl, ethyl or

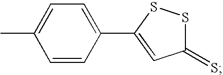

### Z=H;

### X=H;

provided that at least one of R and R′ contain a sulfur atom,
and salts thereof.

Preferred compounds are:
2-(5-[1,2]dithiolan-3-yl-pentanoyloxy)-benzoic acid,
2-acetoxy-benzoic acid 4-(thioxo-5H-[1,2]dithiol-3-yl)-phenyl ester,
2-(5-[1,2]dithiolan-3-yl-pentanoyloxy)benzoic acid ethyl ester,
2-hydroxybenzoic acid 4-(thioxo-5H-[1,2]dithiol-3-yl)-phenyl ester.

In obesity/insulin resistance/metabolic syndrome and its associated complications, multiple mechanisms play a pathogenic role. Some of these mechanisms that interact in its pathogenesis will be briefly described here below. This description is not intended to be exhaustive, and present the definitive pathogenic picture, but rather to show one of its main characteristics, i.e. the complexity and multi-factorial nature of the metabolic syndrome. There is a plethora of information indicating that in insulin resistant states, oxidative stress, via increased production of Reactive Oxygen Species/Reactive Nitrogen Species (ROS/RNS), is involved in the pathogenesis of impaired glucose uptake in insulin's target tissues, and may also play an important part in the pathogenesis of the cardiovascular complications (endothelial dysfunction, atherosclerosis) commonly associated with this condition.

Another important pathogenic mechanism is inflammation, originating in adipose tissue. Augmented synthesis of pro-inflammatory cytokines by adipose tissue, such as tumour necrosis factor-α, activates the ubiquitous nuclear transcription factor κB (NF-κB). NF-κB activation, in turn, induces oxidative stress as well as the formation of additional inflammatory cytokines, and sets up a vicious cycle. Finally, over the past few years, evidence has emerged that gasotransmitters, such as nitric oxide, carbon oxide and hydrogen sulfide play a key role in the regulation of tissue homeostasis in experimental cardiovascular and metabolic experimental conditions.

The pharmacological agents described in the present invention are capable of interacting with each of the above-mentioned major pathogenic mechanisms. The first objective is to act directly on the “radical shower” produced by oxidative stress, by restoring the balance between ROS/RNS synthesis and the antioxidant defences.

Enhanced formation and accumulation of advanced glycation endproducts (AGEs) have been implicated as a major pathogenesis process leading to diabetic complications, normal aging, atherosclerosis etc. The compounds of the present invention act also as AGE inhibitors.

Among the substances that theoretically can be utilized to interfere with the cellular stress cascade, gasotransmitters such as NO or H₂S occupy a prominent role. However, important aspects need to be taken into account. First, although gasotransmitters are capable to inhibit effectively oxidative stress, they are themselves often present as radicals, particularly when massively released, and may therefore interact with ROS/RNS. Moreover, blocking the transcription factors involved in oxidative stress may also impair anti-oxidant defense mechanisms. To circumvent these deleterious effects, the release of gases by gasotransmitters needs to be controlled, and mimic the slow and sustained release produced by the endogenous enzymes. Thus, slow releasing gasotransmitters have the potential to reduce oxidative stress and restore nitric oxide homeostasis, two major pathogenic mechanisms of the metabolic syndrome and its associated complications. The physico-chemical characteristics of the gasotransmitter are another important aspect that needs to be considered.

It has been found that (acetyl) salicylic derivatives of lipoic acid are useful for the treatment of metabolic syndrome. Lipoic acid is a coenzyme in the oxidative decarboxylation of α-keto acids and is found in virtually every cell in the body. The antiinflammatory, analgesic and cytoprotective properties of lipoic acid, and its antioxidant effect, make it an interesting active ingredient for pharmacy, cosmetics, food science and adjacent areas (Biothiols in Health and Disease, edited by Packer L. and Cadenas E., Marcel Dekker Inc., New York, Basle, Hong Kong). Thus, studies on diabetic patients in which administration of lipoic acids showed an effect have been reported. For example, Jacob et al., Arzneim.-Forsch./Drug Res. 45 (II) No. 8 (1995) 872-874 describe a distinct improvement in the glucose utilization of patients with type II diabetes after a single parenteral dose of 1,000 mg of lipoic acid.

Similar results have been reported with chronic parenteral administration (Jacob et al., Exp. Clin. Endocrinol. Diabetes 104 (1996) 284-288). In a study of the treatment of diabetic neuropathy with lipoic acid (ALADIN) symptomatic complaints decreased with intravenous administration of 600 mg of lipoic acid a day for 3 weeks (Ziegler et al., Diabetologia (1995) 38: 1425-1433).

It was found in a recent multicenter study of patients with type II diabetes that oral administration of 600 mg of lipoic acid once to 3 times a day was able to influence the insulin sensitivity (Jacob et al., Free Radical Biology & Medicine, Vol. 27, Nos. 3/4, 309-314, 1999 and BioFactors 10 (1999) 169-174).

However also lipoic acid has some relevant drawbacks, being absorbed by oral route only in a partial and erratic way.

It has been found, that the compounds of the present invention are capable to interfere with the most of the major pathways involved in the pathogenesis of insulin resistance/metabolic syndrome and its associated complications. These derivatives showed not only a remarkable safety but also an improved potency.

When the compounds include at least one asymmetric carbon atom, the products can be used in racemic mixture or in form of single enantiomer.

The compounds of the present invention are more effective and safer and can be used also in the other therapeutic indications that are associated directly or indirectly with the metabolic syndrome (and thereby to inflammation, on the basis of what said above) in the cardiovascular system (for example myocardial and vascular ischemia in general, hypertension systemic and regional, stroke, atherosclerosis, etc), connective tissue disease (for example arthritis and connected inflammatory diseases, etc), respiratory system (for example asthma, COPD, etc.), gastrointestinal system (for example ulcerative and non-ulcerative diseases, intestinal inflammatory diseases, liver cirrhosis, etc) urogenital system (for example impotence, incontinence, etc.), central nervous system (Alzheimer disease, Parkinson's disease and neurodegenerative diseases in general), cutaneous system (eczema, neurodermatitis, acne, etc.), infectious diseases (of bacterial, viral, parasitic origin) and for chemotherapy in different organs (colon, lung, prostate, ovaries, uterus, breast, tongue, liver, bone, etc.), in prevention and/or treatment as monotherapy or in association with other cytostatic agents or radiotherapy.

The compounds of the present invention can be used for all the therapeutic indications where the parent compound is indicated or even suggested in public documents and all the pathologies where metabolic syndrome plays a direct or indirect pathogenetic role.

Furthermore, a relatively better water solubility of the compounds described in the present invention in principle guarantees a better absorption from the GI (gastro intestinal) tract and consequently a better potency.

Also the possibility to prepare parenteral formulations, due to salification of the carboxylic ending, is an advantage for some of these anti-inflammatory compounds and it is part of the present invention.

Pharmaceutical acceptable salts, such as for example salts with alkaline metals and alkaline earth metals, non-toxic amines and aminoacids, are also part of the present invention. Preferred salts are the salts with arginine and agmatine.

Depending on the specific condition or disease state to be treated, subjects may be administered compounds of the present invention at any suitable therapeutically effective and safe dosage, as may be readily determined within the skill of the art. For example, compounds of the present invention may be administered at a dosage between about 1 and 90 mg/kg, and more preferably between about 3 and 30 mg/kg.

It is a further object of the present invention a method for treating inflammatory diseases, comprising administering to a patient in need thereof a therapeutically effective amount of a compound of general formula (I). More in particular a method where the inflammatory disease is metabolic syndrome, diabetes, obesity, dyslipidemia, insulin resistance.

As a further preferred embodiment of the method for treating inflammatory diseases the compound of general formula (I) is administered at a dose of about 10 mg to about 1 g.

The compounds of the present invention can be administered in the form of any pharmaceutical formulation, the nature of which will depend upon the route of administration. These pharmaceutical compositions can be prepared by conventional methods, using compatible, pharmaceutically acceptable excipients or vehicles. Examples of such compositions include capsules, tablets, syrups, powders and granulates for the preparation of extemporaneous solutions, injectable preparations, rectal, nasal, ocular, vaginal etc. A preferred route of administration is the oral and rectal route.

The following non-limitative examples further describe and enable an ordinary skilled in the art to make and use the invention.

### EXAMPLE 1

### Synthesis of 2-(5-[1,2]Dithiolan-3-yl-pentanoyloxy)-benzoic acid

The acylchloride of lipoic acid was prepared adding to a solution of lipoic acid (3.36 mmoles) in 4 ml dry dichloromethane, an equimolar amount of oxalyl chloride. The solution was stirred for 4 hours at 0° C., and then the solvent was removed under reduced pressure.

Then 3.39 mmol of salicylic acid were added to a solution of lipoic acylchloride in dry THF. 3.39 mmol of diisopropylethylamine were added dropwise and the solution was stirred for 3 hours at room temperature. The solvent was evaporated and the residue was dissolved in dichloromethane, extracted with 1M HCl and the organic phase was dried on anhydrous sodium sulphate and evaporated. The obtained residue was chromatographed on silica gel eluting with dichloromethane.

### EXAMPLE 2

### Synthesis of 2-acetoxy-benzoic acid 4-(thioxo-5H-[1,2]dithiol-3-yl)-phenyl ester

To 280 mmol of sulfur, 40 mmol of anethole were added. After heating at 200° C. for 6 hours, 2.5 g of anethole dithiolethione were obtained. The product, washed with ether, was crystallized by ethyl acetate: melting point 110-111° C. Then 1.5 g of anethole dithiolethione were mixed with 7.5 g of pyridine HCl and the mixture was heated for 25 minutes at 215° C. After cooling, 1N HCl in excess was added and the precipitate was filtered, washed and crystallized from ethanol. The obtained compound melted at 191-192° C.

The ester of acetyl salicylic acid with 5-(4-hydroxyphenyl)-3H-1,2-dithiol-3-thione was prepared via the acyl chloride of acetyl salicylic acid. 5-(4-hydroxyphenyl)-3H-1,2-dithiol-3-thione and N-(Et) (iPr)₂ (0.62 ml) were added to a solution of acylchloride of acethyl salicylic acid (3.5 mmoles) in dry THF and the mixture was refluxed for 6 hours under nitrogen.

After removal of THF, the mixture was dissolved in dichloromethane, washed with 0.25 M HCl followed by water and finally by 0.1 N NaOH. After evaporation of the solvent, the residue was chromatographed on silica gel eluting with dichloromethane/ciclohexane (8/2). The compound was crystallized from ethanol and showed a melting point of 128-129° C.

### EXAMPLE 3

### Synthesis of 2-(5-[1,2]Dithiolan-3-yl-pentanoyloxy)-benzoic acid ethyl ester

A 1N solution of dicyclohexylcarbodiimide (DCC) (1,100 g, 5.3 mmol) in dichloromethane was added to a solution of ethyl salicylate (645 mg, 3.88 mmol), lipoic acid (1,050 g, 5 mmol) and dimethylaminopyridine (DMAP) (20.5 mg) in 50 ml of anhydrous dichloromethane.

The mixture was stirred at room temperature for 3 hours under nitrogen. At the end of the reaction, the resulting mixture was filtered and the solution was evaporated and the residue chromatographed on silica gel eluting with dichloromethane/cyclohexane (1/1).

The resulting product was a viscous yellow oil with an yield >90%.

### EXAMPLE 4

### Pharmacological Test

The effects of administration of 50 mg/kg/day of the compound of Example 2, by gavages to high-fat diet fed insulin resistant mice [Cook et al. Diabetes, 53:2067-72, 2004] for 7 days, in terms of glucose infusion rate (mg/kg per min, during the steady state phase of hyperinsulinemic euglycemic clamp studies) were reported in the following table 1:

TABLE 1

| Placebo (1) | Placebo (2) | Compound Example 5 |
|---|---|---|
| 60.6 | 69.8 | 86.4 |

With P < 0.01 (n = 8)

### EXAMPLE 5

### Synthesis of 2-hydroxybenzoic acid 4-(thioxo-5H-[1,2]dithiol-3-yl)-phenyl ester

To 280 mmol of sulfur, 40 mmol of anethole were added. After heating at 200° C. for 6 hours, 2.5 g of anethole dithiolethione were obtained. The product, washed with ether, was crystallized by ethyl acetate: melting point 110-111° C. Then 1.5 g of anethole dithiolethione were mixed with 7.5 g of pyridine HCl and the mixture was heated for 25 minutes at 215° C. After cooling, 1N HCl in excess was added and the precipitate was filtered, washed and crystallized from ethanol. The obtained compound melted at 191-192° C.

To a solution of 424 mg (3.09 mmol) of salicylic acid, 700 mg (3.09 mmol) of 5-(4-hydroxyphenyl)-3H-1,2-dithiol-3-thione and 379 mg (3.09 mmol) of 4-dimethylaminopyridine in 70 ml of dichoromethane a 1 N solution of dicyclohexylcarbodiimide (DCC) in CH₂Cl₂ (3.40 mL) were added. The mixture was stirred at room temperature and under N₂ for 3 h. After filtration of the dicyclohexylurea (DCU), the solution was extracted first with 1N HCl (64 ml), then with brine (64 ml) and finally with a saturated solution of NaHCO₃. The solution was evaporated to dryness and the obtained residue was chromatographed on silica gel using a mixture of CH₂Cl₂-cyclohexane (6:4) as eluent. The compound, after washing with ether, showed a m.p. of 177.178° C. (yield 53%).

### EXAMPLE 6

### Biological Data

It is widely known that metabolic syndrome is generally accompanied by oxidative stress. Also in the paper of Ogihara (Ogihara T. et al. “Oxidative stress induces insulin resistance by activating the nuclear factor-kB pathway and disrupting normal subcellular distribution of phosphatidylinositol 3-kinase” in Diabetologia 47, 794-805 (2004) such relationship was found. The effect of oxidative stress-induced hypertension in rats has been evaluated in male Wistar rats (10 animals/group). Hypertension was induced by oral administration of buthionine sulphoximine (BSO) (30 mmol/L/day) to the drinking water for seven days.

Compounds of examples 2, 3 and 5 suspended in the vehicle at the dose of 50 mg/kg/day were administered for seven days and some parameters measured in the experiments are reported in table 2.

TABLE 2

| | | BSO + | BSO + | BSO + | BSO + |
|---|---|---|---|---|---|
| | Vehicle | Vehicle | Cpd Ex 5 | Cpd Ex 6 | Cpd Ex 8 |
| % Increase | 0% | 80% | 10% | 50% | 15% |
| systolic blood | | | | | |
| pressure | | | | | |
| % Plasma | 0% | 65% | 5% | 20% | 7% |
| glutathione | | | | | |
| decrease | | | | | |
| Endothelium | 95% | 25% | 88% | 60% | 80% |
| function (% | | | | | |
| contraction | | | | | |
| induced by | | | | | |
| acetylcholine) | | | | | |

Vehicle: Carboxymethylcellulose 0.5% (w/w) in water

1. Compounds of general formula (I): 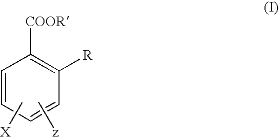
wherein:
R=—OCOCH₃, —OH, 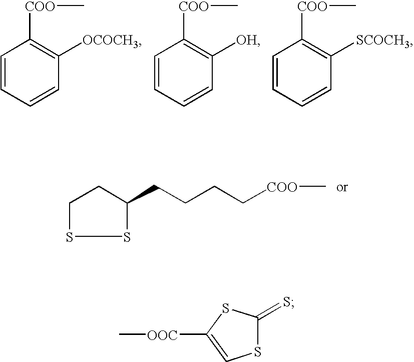
R′ is H, methyl, ethyl or 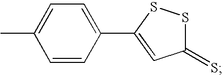
Z=H;
X=H;
provided that at least one of R and R′ contain a sulfur atom,
and salts thereof.

2. A compound according to claim 1, wherein said compound is 2-(5-[1,2]dithiolan-3-yl-pentanoyloxy)-benzoic acid.

3. A compound according to claim 1, wherein said compound is 2-acetoxy-benzoic acid 4-(thioxo-5H-[1,2]dithiol-3-yl)-phenyl ester.

4. A compound according to claim 1, wherein said compound is 2-(5-[1,2]dithiolan-3-yl-pentanoyloxy)benzoic acid ethyl ester.

5. A compound according to claim 1, wherein said compound is 2-hydroxybenzoic acid 4-(thioxo-5H-[1,2]dithiol-3-yl)-phenyl ester.

6. A compound of claim 1 salified with arginine.

7. A compound of claim 1 salified with agmatine.

8. Use of a compound according to claim 1 for the manufacture of a medicament for treatment of inflammatory diseases.

9. Use according to claim 8, wherein the inflammatory disease is metabolic syndrome, diabetes, obesity, dyslipidemia, insulin resistance.

10. Pharmaceutical compositions for treating inflammatory diseases, comprising a compound according to claim 1 at a dose from 10 mg to 1 g.

11. Pharmaceutical compositions for treating inflammatory diseases, comprising a compound according to claim 1 as well pharmaceutically acceptable adjuvants and/or carriers.

12. Pharmaceutical compositions comprising at least one compound according to claim 1 as an active component.
